(19) United States
(12) Patent Application Publication
(10) Pub. No.: US 2023/0149879 A1
(43) Pub. Date: May 18, 2023

(54) REACTION DEVICE, MICROSPHERE PREPARATION DEVICE AND EXTRACTION METHOD AND LIPOSOME DRUG LOADING METHOD

(71) Applicant: BIODOSAGE TECH CO., LTD., Shanghai (CN)

(72) Inventor: Tuo Jin, Shanghai (CN)

(73) Assignee: BIODOSAGE TECH CO., LTD., Shanghai (CN)

(21) Appl. No.: 17/916,576

(22) PCT Filed: Apr. 11, 2021
(86) PCT No.: PCT/CN2021/086338

§ 371 Date: Oct. 2, 2022

(30) Foreign Application Priority Data
Apr. 10, 2020 (CN) 202010281169.4

(51) Int. Cl. B01J 13/20; B01J 14/00; B01J 19/06; B01J 4/00; A61K 9/127; A61K 9/16

(52) U.S. Cl. CPC B01J 13/206; B01J 14/00; B01J 19/06; B01J 4/001; A61K 9/1277; A61K 9/1682; B01J 2204/002

(57) ABSTRACT

Disclosed is a reaction device, comprising: a reactor body (100) and a supply device (200), wherein the reactor body (100) has a first end (106) and a second end (107) and is used for accommodating a reaction liquid, with a first injection port (101) being provided between the first end (106) and the second end (107), and a discharge port (109) being provided at the second end (107); and the supply device (200) is in communication with the first injection port (101) to inject a continuous phase, wherein the continuous phase directionally flows in the reactor body (100) to form or maintain a parameter gradient in the reactor body (100). By means of injecting the continuous phase into the first injection port (101) on the reactor body (100), the solution presents a certain parameter gradient on two sides of the first injection port (101) in the reactor body (100).

### TECHNICAL FIELD

The invention relates to the field of pharmacy, in particular to a reaction device, a microsphere preparation device and an extraction method, and a liposome drug loading method.

### BACKGROUND

Microspheres are tiny spherical particles with particle sizes ranging from 1 to 250 μm. Polymer microspheres, having been extensively studied since the 1970s, are regarded with high potential for therapeutic drugs because of their excellent fluidity, ease of injection, and slow release of the encapsulated components. The concept was first proposed from the article “Polymers for sustained release of proteins and other macromolecules” published by R. Langer and J. Folkman in the journal Nature (263:793-800). In this journal article, in view of the fact that biological drugs have outstanding therapeutic effect but are difficult to be absorbed through tissue membranes while being taken orally, and so they have to been administered frequently by injection, the author proposes to encapsulate the biological drugs into biodegradable polymer microspheres, to achieve long-acting and sustained-release.

The market for recombinant protein drugs has soared at an annual growth rate of 14-16% since the 1980s and now exceeds 50% of all prescription drugs. At present, there are more than 230 protein and peptide drugs approved for sales, and another 9,000 are in the R&D pipeline, and some of these R&D products may be launched in the next few years. In stark contrast to the rapid growth of biological drugs, the way to deliver these drugs remain the same, by frequent injections, and thus drug delivery technology calls for urgent breakthroughs.

Long-acting injections and high-efficiency non-injection biological drugs, alternatives to frequent injections, are two easy-to-understand formulations, which have attracted decades of R&D investments by those skilled in the art. So far, there has been no breakthrough in the high-efficiency non-injectable formulation of biological drugs; whereas a series of long-acting injection products have been ready for the markets. The long-acting injections of biopharmaceutical drugs on the market adopt two kinds of schemes: the method of prolonging a half-life in vivo by chemical modification (PEGylation) or biological modification (changing the peptide sequence or protein fusion) and the slow release of drug depots at the injection sites. The former is limited by the mechanism that the in vivo concentration of the modified molecule decreases exponentially after injection, and the drug efficacy generally only lasts for one week, with the longest does not exceed two weeks, or few drugs with a wide therapeutic window, by a large dose, to increase the drug efficacy to four weeks. Further the specific activity is reduced due to the shielding effect of the modifying group, and hence the dosage must be increased. The latter can theoretically maintain the drug efficacy of a single injection for weeks or even months, but it is only successful in microsphere injections, and there are currently only 8 drugs using long-acting sustained-release microsphere preparations (excluding two contrast agents).

Why, biological drugs, such as peptides, which can only be administered by injection, are experiencing significant growth, whereas, microspheres, as the only drug preparation that can achieve long-term effects for several weeks, counterintuitively, are only used in extremely limited drugs? The only key reason lies in the cumbersome and difficult-to-reproduce production process for microsphere formulations. The current production process of microsphere preparations in the pharmaceutical industry includes two types: double emulsification method and silicone oil phase separation method. The steps of the double emulsification method include: firstly, emulsifying and dispersing the polypeptide aqueous solution in the organic solution of the biodegradable polymer, and then further emulsifying and dispersing the formed “water-in-oil” emulsion in the continuous phase of the polyvinyl alcohol aqueous solution to form a “complex emulsion”. Lastly, the organic solvent in it is extracted under reduced pressure to solidify the polymer dispersed phase into spheres, but in actual use, the solution used for extracting the organic solvent is often saturated, and the extraction cannot be carried out any more, and continuous production cannot be formed.

As disclosure herein, continuous production can be realized by improving the preparation device.

### SUMMARY OF THE INVENTION

The invention discloses a reaction device, a microsphere preparation device, an extraction method, and a liposome drug-carrier method, and solves the technical problem that during the existing microsphere production process, specifically at extraction step the solution as an extraction agent becomes easily saturated and cannot achieve a continuous production.

The technical scheme disclosed by the present invention is described in detail below.

A reaction device, comprises a reactor body, having a first end and a second end, configured to enclose a first liquid, and a first injection port is provided between the first end and the second end of the reactor body, and a discharge port is provided at the second end of the reactor body.

Said reaction device further comprises a supply device, in communication with the first injection port, for injecting a continuous phase, wherein the continuous phase flows directionally within the reactor body to create or maintain a parametric gradient within the reactor body. The continuous phase refers to a liquid that surrounds dispersed substances and is in a continuous state. In embodiments of the extraction process, the continuous phase refers to the extraction agent.

In the present invention, in one embodiment, by providing a first injection port between the first end and the second end of the reactor body, the supply device injects the continuous phase into the first injection port, and the continuous phase flows in a preset direction within the reactor body such that to have a parameter gradient within the reactor, and there is a concentration difference of the extracted substances in the continuous phase on both sides of the first injection port in the reactor body along the preset direction. The concentration difference arises in the detail implementation of the extraction as follows. The substance to be treated is injected from the first end of the reactor body, and the continuous phase is used as an extraction agent. Since the continuous phase is injected from the first injection port, the flow rate of the continuous phase from the first injection port to the second end is greater than the flow rate of the continuous phase from the first end to the first injection port, causing concentration of the extracted substances in the continuous phase from the first end to the first injection port is greater than that in the continuous phase from the first injection port to the second end. Therefore, there is a difference in the concentration of the extracted substance in the continuous phase on both sides of the first injection port. In one detail embodiment of organic solvents in extraction microspheres, the continuous phase is water, and the substance to be treated is embryo microspheres, and the organic solvents in embryo microspheres such as dichloromethane, ethyl acetate, acetic acid, ethanol, etc., are extracted out by water. An exemplar concentration difference, at 25° C., the concentration of the extracted dichloromethane in water between the first end and the first injection port is 0.5-1.1 wt %, and the concentration between the first injection port and the second end is 0.01-0.49 wt %.

Preferably, the reactor body is positioned obliquely or vertically, the first end is located above the second end; the continuous phase flows directionally towards the second end under the force of gravity.

According to the aspects of the present invention, when the reactor body is placed vertically, the continuous phase between the first end and the first injection port is static with respect to each other, and the continuous phase between the second end and the first injection port flows towards the second end and through the discharge port, thereby forming a chemical parameter distribution gradient between the different stages of the continuous phase. The finished products, such as microspheres or liposomes, pass through of different gradients of chemical parameters, the chemical parameters of the different gradients can be enumerated as there are, concentration differences of organic solvents in the continuous phase, temperature differences of continuous phase or pH differences of continuous phase at different locations, when moving from the first end to the second end, so that the products can meet the ideal physicochemical environment throughout all stages of their formation.

Migration of product particles requires a driving force. In a vertical or inclined reaction device, product particles, such as microspheres or liposomes, can be driven by gravity to settle, the flow rate of the continuous phase at the first end is slower than that of the continuous phase at the second end, so that the gradient of the chemical parameters is maintained.

Alternatively, the body of the reactor is placed horizontally, and the first end is provided with a second injection port for injecting a treatment liquid with an initial flow rate; the continuous phase is driven by the treatment liquid toward the second end in a preset direction.

In this embodiment, when the body of the reactor is placed horizontally, horizontally herein refers to the direction perpendicular to the direction of gravity of the Earth, the migration of product particles from the first end to the second end depends on the overall flow of the continuous phase. The flow rate of the continuous phase between the first end and the first injection port is slower than the flow rate of the continuous phase between the first injection port and the second end, the continuous phase between the second end and the first injection port is continuously injected with fresh continuous phase at the first injection port and the discharge port discharged continuously. In the case of discharge, the flow rate is significantly greater than the flow rate of the continuous phase between the first end and the first injection port; thereby maintaining a gradient in the chemical parameters of the continuous phase composition.

Preferably, the first injection port is arranged in the middle between the first end and the second end of the reactor body.

In this embodiment, by arranging the first injection port between the first end and the second end of the reactor body, and located in the middle of the reactor main body, between the two sections located in the middle of the first end and the second end of the reactor body. The first injection port is located in the reactor body. The parameter gradient distribution of the continuous phase components is easily formed on both sides of the injection port; the distribution of the parameter gradient can be optimized by adjusting the position of the first injection port.

Preferably, the reactor body comprises
a first accommodating portion and a second accommodating portion;
a first joint, connecting the first accommodating portion and the second accommodating portion, one end of the first accommodating portion away from the first joint is the first end, and the first injection port is disposed in or on the first connector joint;
a second joint communicates with the second accommodating portion to form the second end, and a discharge port is disposed on the second joint.

In this embodiment, the end of the first accommodating portion away from the first joint is a top end of the first accommodating portion, which is also the top end of the entire reactor body, and the first injection port is disposed on a surface of the first joint. The second joint is arranged at the end of the second accommodating portion away from the first joint, an end of the second accommodating portion close to the first accommodating portion is a top end, and another end away from the first accommodating portion is the distal end, and serves as the overall discharge port of the reactor body. The reactor body includes the first accommodating portion, second accommodating portion, the first joint and the second joint, so that they can be installed as needed during use and can be assembled by using the first joint or the second joint according to the needs. When it is not needed, it can be disassembled, cleaned, and even sanitized, making it easy for transportation.

Preferably, said reaction device is an embryonic microsphere solidification reactor or a liposome drug-carrier reactor.

A device for preparing microspheres, comprises the following structural components.

The material supply injection mechanism is used to output embryo microspheres. In any one of the above reaction devices, the first end of the reaction device is provided with the third injection port, which is communicated with the material injection mechanism, and the embryo microspheres are injected into the reaction device through the material injection mechanism, so that the embryonic microspheres are settled in the reaction device and solidified by extraction to form microspheres.

In this embodiment, the material injection mechanism is used to output embryonic microspheres, regulate the particle size of the microspheres, prevent the leakage of the microsphere carrier, and avoid the denaturation and inactivation of the biologically active carrier.

The top of the reaction device is provided with a material injection mechanism, and the material is the raw material for forming the embryonic microspheres, including the matrix of the microspheres, the carrier, and the auxiliary materials for regulating the performance of the microspheres; the material injection mechanism is: a component for converting the material solution into embryonic microspheres, and injecting the shaped embryonic microspheres into the continuous phase from the top of the first accommodating portion.

The injected embryonic microspheres move from the top to the end of the reactor body, that is, from the first end to the second end, passing through the continuous phase distributed according to the designed concentration gradient, before or while reaching the end of the reactor. Solidification due to solvent extraction to form microspheres.

The collector can also be designed as an independent portion communicated with the discharge port, the discharge port is connected with the top end of the collector through the connection port, and the end of the collector becomes the end or bottom end of the whole reactor or preparation device.

The granular products such as microspheres or liposomes have different requirements for the concentration of different components of the continuous phase during their molding, curing, or aging, drug loading, etc., and in order to meet these requirements, the concentration must be within the desired gradient profile is achieved in the reactor body. For example, when the embryonic microspheres leave the injection mechanism and enter the first accommodating portion, the organic solvent in the continuous phase needs to be close to saturation, so that the embryonic microspheres can be formed in a leisurely manner, avoiding the rapid extraction of the solvent which can lead to microsphere forming polymers or other materials and get precipitated and attached at the outlet of the injection mechanism; when the embryo microspheres are formed and separated from the material injection mechanism and enter the curing process, the organic solvent in the continuous phase needs to be as low as possible to improve the curing efficiency. The preparation of liposomes also has the same requirement for continuous phase concentration gradient distribution. For example, the drug loading stage needs a pH that is favorable for the drug to be liposoluble, while the dispersion or storage process after drug loading needs to be continuous phase that is conducive to the drug being non-lipid soluble pH.

In one aspect, the present invention discloses a microsphere preparation method, which comprises the steps of forming the embryonic microspheres in the material injection mechanism, so that the embryonic microspheres are moved in the reaction device , and at the same time they are solidified when encounter the extraction of the organic solvent, and a collector collects the hardened embryonic microspheres and transfers them to washing process to further remove unwanted impurities from the microspheres and from the continuous phase.

In another aspect, the present invention discloses a microsphere solidification method, which includes the steps of extracting an organic solvent out of the embryonic microspheres, and steps can be implemented based on any one of the above-mentioned reaction devices, wherein the first liquid is a microsphere receiving solution, and the microsphere receiving solution comprises water as a matrix, to extract the organic solvent out of the embryonic microspheres.

Moving the embryonic microspheres from the first end to the second end along the reactor body containing the microsphere receiving liquid, extracting the organic solvent out of the embryonic microspheres, and the embryonic microspheres harden to form microspheres.

During the extraction, water is injected through the first injection port, and the water flows to the second end in the reactor body, so as to form a concentration gradient of organic solvents extracted out of the embryonic microspheres in water on both sides of the first injection port.

In the present invention, in one embodiment, by injecting water without organic solvent into the first injection port, the organic solvent extracted out of the embryonic microspheres is continuously carried away with the flow of water, so that the organic solvent on the surface of the embryonic microspheres can be extracted continuously. The organic solvent extracted out of embryonic microspheres can be dichloromethane, ethyl acetate, ethanol, methanol, acetic acid. In this embodiment, the continuous phase is water, the water flows toward the second end of the reactor body, and a concentration difference occurs from the two sides of the first injection port at the dispersed phase in the reactor body, so that the solution in the reactor body is in an unsaturated state, and the concentration of the solution located above is greater than that below. Take dichloromethane as the organic solvent extracted from embryo microspheres as an example. The concentration range of the dichloromethane in the water on the side of the first injection port close to the first end of the main body of the reactor is 1-2 wt %. Since the flow rate of water from the first injection port to the second end of the reactor body is greater than the flow rate of water from the first injection port to the first end of the reactor body, the concentration of dichloromethane in the water on the side of the first injection port close to the second end of the reactor body is less than 1 wt %. The first dispersion refers to the organic solvent being carried away by the flowing water. The second dispersion refers to organic solvents extracted out of embryonic microspheres in the continuous phase. The concentration of the solution is such that the embryo microspheres are emulsified in the high concentration solution, and the embryo microspheres are extracted in the low concentration solution as the embryo microspheres flow toward the second end, so that embryo microsphere stock solution, continuously injected at the first end, can be continuously extracted, so that the embryo microsphere can be well formed. Similarly, by injecting a continuous phase containing a certain organic component into the first injection port, a concentration gradient distribution of the component in the continuous phase is formed during flow and diffusion process.

Although the continuous phase in the first accommodating portion does not take part in the flow of the continuous phase in the second accommodating portion, when the organic solvent contained in the continuous phase in the first accommodating portion diffuses into the second accommodating portion with a low concentration to form a certain concentration gradient. The organic solution in the continuous phase of the first accommodating portion comes from extraction from embryonic microspheres or manually added.

Preferably, the water can be injected continuously or intermittently; and/or

directional flow of water is achieved by gravity or by the flow of microsphere-receiving fluid.

In this embodiment, the continuous phase injected into the first injection port continuously or intermittently is water, so that the water can better extract the organic solvent in the embryo microspheres. Using water as the solvent for extraction, is environmental friendly on one hand and is easy to clean on the other hand, for subsequent cleaning of the reactor body. In the process of preparing microspheres, the flow of water can create a concentration gradient distribution of organic solvents or other components in the continuous phase, and can also be used as a driving force to push the microspheres or embryonic microspheres moving from the first end to the second end, especially when the reactor body is positioned horizontally, horizontally here refers to the direction perpendicular to the direction of gravity of the earth.

Preferably, the concentration gradient distribution of the organic solvent or other components in the continuous phase can be achieved by adjusting the flow rate of the continuous phase injected through the first injection port.

In this embodiment, the flow rate of the continuous phase injected by the first injection port can be used to control the distribution of the concentration gradient of the organic solvent or other added components, and at the same time control the preparation rate of microspheres or other granular products.

A liposome drug-carrier preparation method is implemented based on any one of the reaction devices described above. A phospholipid membrane is installed at the first injection port, and a continuous phase is injected into the phospholipid membrane to maintain a pH gradient on both sides of the phospholipid membrane.

Compared with the prior art, the reaction device, the microsphere preparation device and the extraction method, and the liposome drug loading method provided by the present invention have the following beneficial effects.

1. In the present invention, a first injection port is provided between the first end and the second end of the reactor body, so that when the continuous phase is injected into the first injection port, on both sides of the first injection port in the reactor body, a certain concentration gradient or parameter gradient distribution of the substance in solution within the reactor body is formed. For the preparation of microspheres, the formation of a concentration gradient of the organic solvent extracted out of the embryonic microspheres in the continuous phase is beneficial to the formation of embryonic microspheres, which can avoid the precipitation of materials caused by excessive solvent extraction and the adhesion and blockage of the injection mechanism. For lipid loading, it is easier to form a pH gradient, which facilitates the formation of liposome drug-carrier.

2. The flow rate adjustment and optimization of the continuous phase injected through the first injection port can achieve the continuous production of granular products, such as microspheres or liposomes. The flow rate adjustment and optimization can not only deliver a reproducibility of the products, but also it by using small devices it enables large scale production.

3. In the present invention, the reactor body is designed as multi-component set up to facilitate disassembly, installation, and sterilization. Further, to meet a specific user-case scenario, a variety of the components can be assembled for design purpose, and after the microsphere being made, the components can be disassembled and cleaned up as needed.

### DESCRIPTION OF DRAWINGS

The preferred embodiments will be described below in a clear and easy-to-understand manner with reference to the accompanying drawings, and the above-mentioned characteristics, technical features, advantages and implementation methods of the reaction device, the microsphere preparation device and the extraction method, and the liposome drug-loading method will be further described and illustrated.

FIG. 1 is a schematic diagram of an embodiment of the present invention;

FIG. 2 is a schematic diagram of another embodiment of the present invention;

FIG. 3 is a schematic diagram of another embodiment of the present invention;

FIG. 4 is a schematic structural diagram of the microsphere preparation device of the present invention.

Description of elements reference number: reactor body 100, first injection port 101, first accommodating portion 102, second accommodating portion 103, first joint 104, second joint 105, first end 106, second end 107, third injection port 108, the discharge port 109, the supply device 200, the connecting pipe 201, the material injection mechanism 300, and the collector 400.

### DETAIL DESCRIPTION OF EMBODIMENTS

In order to more clearly describe the embodiments of the present invention or difference with respect to the technical solutions in the prior art, the specific embodiments of the present invention will be described below with reference to the accompanying drawings. Obviously, the accompanying drawings in the following description are only some embodiments of the present invention. For those of ordinary skill in the art, other drawings can also be derived from these drawings without significant undue experimentation and method of using the same.

In order to keep the drawings concise, the drawings only schematically illustrate the components related to the present invention, and they may or may not represent its actual structure as a product. In addition, in order to make the drawings concise and easy to understand, in some drawings, only one of the components having the same structure or function is schematically shown, or only one of them is marked. As used herein, “one” not only means “only one”, but also “more than one”.

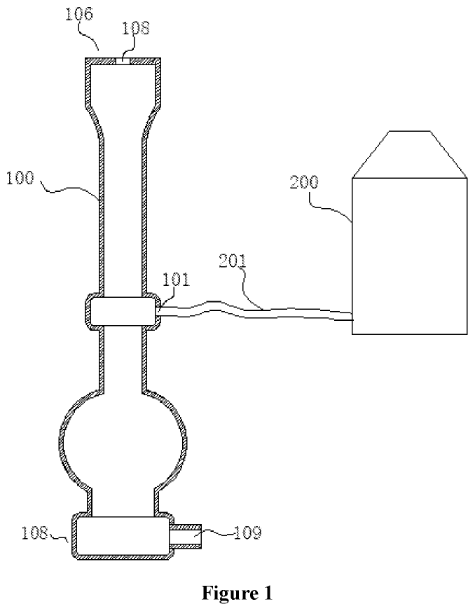

According to an embodiment provided by the present invention, as shown in FIG. 1, a reaction device includes: a reactor body 100 and a supply device 200, the reactor body 100, having a first end 106 and a second end 107, is configured to accommodate the first liquid. A first injection port 101 is provided between the first end 106 and the second end 107, and a discharge port 109 is provided at the second end 107; the supply device 200 is communicated with the first injection port 101 for injecting the continuous phase. Specifically, the supply device 200 is connected with the first injection port 101 through the connecting pipe 201, wherein the continuous phase flows directionally in the reactor body 100 to form or maintain a concertation gradient of the extracted organic solvent in the continuous phase the reactor body 100.

In this embodiment, by providing the first injection port 101 between the first end 106 and the second end 107 of the reactor body 100, so that the supply device 200 can inject the continuous phase into the first injection port 101, and the injected continuous phase, present in the reactor body 100, occurs along a concentration gradient described in detail below. In this embodiment, the concentration gradient of the continuous phase in the reactor body does not depend on the placement position and orientation of the reaction device, nor is it limited to the specific location of the first injection port 101. The reaction device in this embodiment is suitable for microsphere extraction. At the same time, it is also applicable to lipid drug-loaded reactors. In this embodiment, the specific application scenario of the reaction device is including but not limited to the preparation of embryo microspheres and the preparation of drug-loading lipids. The method of using the current reaction device in this embodiment only requires injecting a continuous phase into the first injection port 101 on the reactor body 100. The continuous phase can be used to form or maintain the concentration gradient in the reactor body 100, which is explained in detail in the following specific embodiment. In this embodiment, the first liquid is a mixed solution formed after the continuous phase handing of substances awaiting treatment. Such as in the embodiments of microsphere extraction, the continuous phase is water, the substance of awaiting treatment is embryo microspheres, the mixed solution formed after the continuous phase extracts the organic solvent in the embryo microspheres is the first liquid. The organic solvent extracted out of the embryonic microspheres can be dichloromethane, ethyl acetate, methanol, ethanol or acetic acid. The flowing continuous phase is injected into the first inlet 101, the flow rate of the first liquid between the first injection port 101 and the first accommodating part 102 is slower than that between the first injection port 101 and the second accommodating part 103. Take the organic solvent extracted from embryo microspheres as dichloromethane as an example, as a result, the dichloromethane concentration of the extracted from the embryonic microspheres between the first injection port 101 and the first accommodating part 102 is higher than that of the dichloromethane extracted from the embryonic microspheres between the first injection port 101 and the second accommodating part 103. Therefore, the concentration gradient of the dichloromethane is generated on both sides of the first injection port 101. It should be noted that the concentration gradient formed, in one example, the concentration trend of the gradient is a continuously changing in a linear distribution; in another example, the trend of the concentration gradient is in a non-linear distribution, or a stepwise distribution. In addition, the corresponding parameters within the reaction device may distribute as decreasing from high to low, increasing from low to high, or from high to low then to high again, or from low to high then return to low.

Referring again to FIG. 1, in another embodiment of the present invention, the reactor body 100 is slanted or placed vertically, the reactor is slanted means that the angle formed between the reactor body and the gravity direction of the Earth is greater than 0° but less than 90°, the slanted first end 106 is located above the second end 107; the continuous phase flows towards the second end 107 in a fixed direction under the force of gravity, the first injection port 101 is arranged in the middle, anywhere between one end 106 of reactor body 100 and another end 107 of the reactor body 100, in this embodiment, by arranging the first injection port 101 in the middle of the reactor body, while the reactor body 100 is placed vertically, when forming the embryo microspheres or drug-loading lipids, the concentration gradient in the reactor body 100 changes in a trend because of the continuous phase injected continuously in the middle, which is convenient for the formation of embryo microspheres and the formation of liposome drug carriers. Even when additional injection ports are provided, the weight of the substance injected by the other injection ports is less than the weight of the continuous phase injected by the first injection port, and the concentration gradient of the solution in the reactor body predominately depends on the continuous phase. The vertical placement of 100, when the embryo microspheres or liposomes are injected, they can well settle under the force of gravity, and the continuous phase can flow towards the second end 107, and embryonic microspheres or liposomes then pass through a solution having concentration gradient. The solution can better form embryonic microspheres or liposome-encapsulated drug carriers. In this embodiment, it is not limited to the specific application scenarios of the reaction device, as long as some preparations that meet the requirement of the reaction device, all processes can use the device disclosed herein.

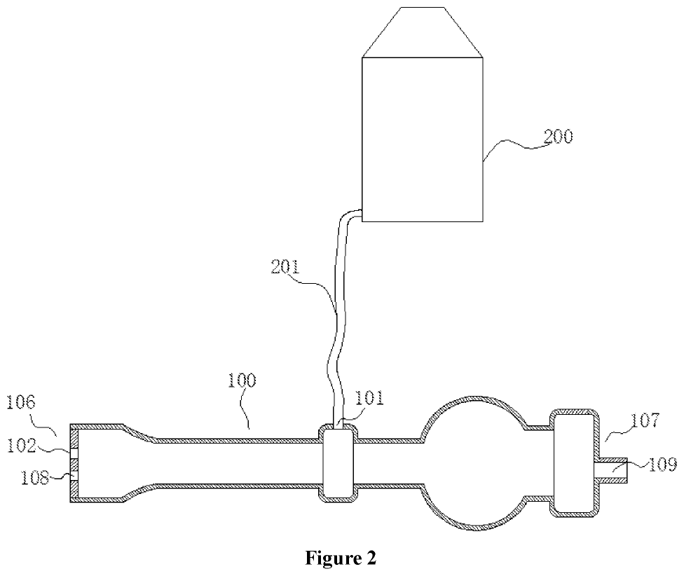

As shown in FIG. 2, in another embodiment of the present invention, the body 100 of the reactor is placed horizontally, and the first end 106 is provided with a second injection port for injecting a treatment liquid with an initial flow rate, in microsphere production, the treatment solution refers to the liquid that helps to form embryonic microsphere and/or the liquid that is used as a continuous phase to extract the embryonic microspheres formed; the first injection port 101 is arranged in the middle, anywhere between the first end and the second end of the reactor body. In this embodiment, the reactor body 100 is placed horizontally, and the first end 106 is injected with a treatment liquid with a certain initial flow rate, so that the continuous phase can flow toward the second end 107, which facilitates the formation of concentration gradients of extracted organic solvent in the continuous phase.

In this embodiment, the provision of the second injection port enables the flow of the liquid inside the reactor body 100 to obtain a movement source, which is more conducive to extraction of microspheres and conducive to the formation of liposome drug-carriers. In this embodiment, when the reaction device is used to extract the microspheres, the treatment liquid can be a continuous phase with a certain initial flow rate, or can be a microsphere receiving liquid with a certain initial flow rate to drive the continuous phase flow.

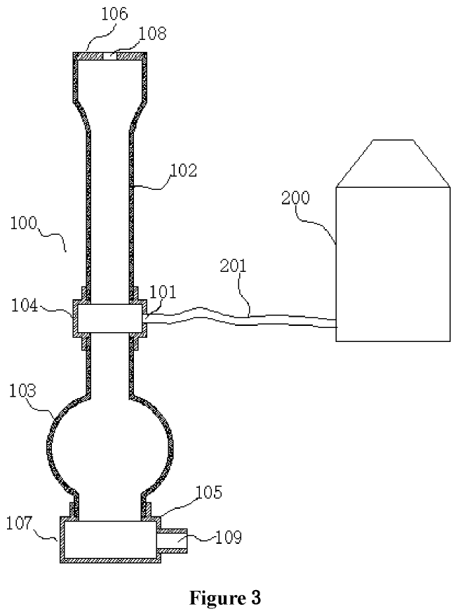

As shown in FIG. 3, in another embodiment of the present invention, the reactor body 100 comprises: a first accommodating portion 102 and a second accommodating portion 103, a first joint 104 and a second joint 105; the first joint 104 is connected to the first joint 104 connects to accommodating portion 102 and a second accommodating portion 103, one end of the first accommodating portion 102 away from the first joint 104 is the first end 106, the first injection port 101 is provided on the first joint 104; the second joint 105 and the second accommodating portion 103 is in communication, becoming the second end 107, further discharge opening 109 is provided on the second joint 105. Specifically, the first accommodating portion 102 and the second accommodating portion 103 are connected to the outer wall of the end of the joint 104, on the outer wall of the end of the joint 104, external thread is provided. Inside of the first joint 104, the inner wall is provided with an internal thread to make the connection between the two, the connection between the first accommodating portion 102 and the first joint 104 more convenient and easier. Similarly, the outer wall of one end of the second accommodating portion 103 connected to the second joint is provided with an external thread, and the inner wall of the second joint is provided with internal threads so that the two, the connection between the second accommodating portion 103 and the first joint 104 are more convenient and easier. In this embodiment, it is not limited to use threaded connection to connects the accommodating portion and joint. In this embodiment, as long as the first joint 104 can be detachably connected to the first accommodating portion 102, the second accommodating portion 103, and the second joint can be detachably connected to the second accommodating portion 103.

It should be noted that the reaction device in the above embodiment is an embryonic microsphere extraction reactor or a liposome drug-carrier reactor, and the opening of the first injection port 101 on the reactor main body 100 makes the reactor body 100 open on the one hand. The concentration gradient of the organic solvent extracted into the continuous phase is formed in the inside of the reactor body, which is beneficial to formation of embryonic microsphere and liposome drug-carrier. On the other hand, the continuous injection of the continuous phase helps to maintain continuous formation of the embryonic microsphere and liposome drug-carrier.

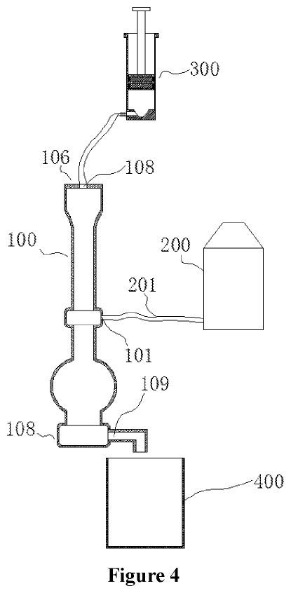

As shown in FIG. 4, a microsphere preparation device comprises: a material injection mechanism 300, the reaction device described in any of the above-mentioned embodiments, and a collector 400. The material injection mechanism 300 is used to output embryonic microspheres, and the first end 106 of the reaction device is provided with a third injection port 108, which is communicated with the material injection mechanism 300. After the embryo microspheres are formed, they fall off from the material injection mechanism 300, settle in the reaction device, and form microspheres through extraction and solidification of the solvent. The collector 400 communicates with the second end 107 of the reaction device or is located at the end of the reactor body 100 as a part of the second end 107 to collect the microspheres, wherein the embryo microspheres formed, by the material injection mechanism 300, are transported into the reactor body 100. The first injection port 101 continuously injects the continuous phase, through the flow of the continuous phase and the diffusion of the organic solvent, a concentration gradient of the organic solvent extracted out of the embryonic microspheres forms a concentration gradient in the continuous phase is formed on both sides of the first injection port 101 in the body 100 of the reactor, the concentration of the organic solvent in the continuous phase between the first end 106 and the first injection port 101 is greater than the concentration of the organic solvent in the continuous phase between the first injection port 101 and the second end 107, and the embryonic microspheres are emulsified in the place where the concentration of the organic solvent is high, in order to improve the reproducibility of sphere formation, avoid the precipitation of polymers or other sphere-forming materials caused by excessive solvent extraction, and the adhesion and blockage of the microsphere forming mechanism. The spheres are solidified at low organic solvent concentration, and the solidified embryo microspheres are accumulated by the collector 400; and then post-processing is carried out in the post-processing equipment. During the post-processing, the microspheres are first rinsed to remove any impurities in the microspheres or in the continuous phase. The undesired residue is then lyophilized on the microspheres. The material injection mechanism 300 has an embryonic microsphere forming structure inside.

A microsphere solidification method for extracting an organic solvent in embryonic microspheres, implemented based on any one of the above-mentioned reaction devices, the first liquid is a microsphere receiving solution, and the microsphere receiving solution uses water as a matrix to extract the organic solvent.

The embryonic microspheres are made to flow from the first end 106 to the second end 107 along the reactor body 100 containing the microsphere receiving liquid, the organic solvent inside of the embryonic microspheres is extracted, and the embryonic microspheres harden to form microspheres.

During extraction, water is injected through the first injection port 101, and the water flows in the reactor body 100 to the second end 107 to form a concentration gradient of organic solvents in water on both sides of the first injection port 101.

Specifically, water can be injected continuously or intermittently; the embryonic microspheres can move directionally by gravity or the flow of the microsphere-receiving fluid. In the specific implementation, the injection mode of the water flow can be controlled as required, and continuous injection or intermittent injection can be selected. The flow rate of the continuous phase injected by the first injection port 101 can be adjusted to control the continuous phase. Or the distribution of concentration gradients of organic solvents or other added components in the continuous phase, while regulating the rate of preparation of microspheres or other granular products. When the reaction device is placed horizontally, the flow of the continuous phase from the reactor body 100 to the second end 107 becomes the driving force for the movement of embryonic microspheres or microspheres, or other granular products. The specific selection can be selected according to end use requirement, and the above-mentioned treatment liquid can be the directional flow microsphere receiving liquid in this embodiment.

A liposome drug-carrying preparation method is implemented based on any of the above-mentioned reaction devices, and the phospholipid membrane is installed at the first injection port 101, and the continuous phase is injected to make the phospholipid membrane maintain a pH gradient on both sides, so that the phospholipid membrane can better encapsulated drugs to form a liposome drug carrier.

It should be noted that the above embodiments can be freely combined as desired. The above are only the preferred embodiments of the present invention. It should be pointed out that for those skilled in the art, without departing from the principles of the present invention, several improvements and modifications can be made, which shall be deemed to be the part of the invention disclosed herein.

1. A reaction device, comprising:
a reactor body, configured to enclose a first liquid, having a first end and a second end,
a first injection port is provided between a first end and second end of the reactor body, and
a discharge port is provided at the second end of the reactor body; and
a supply device, communicated with the first injection port, for injecting a continuous phase; wherein the continuous phase is a liquid that surrounds dispersed substances flows directionally within the reactor body to create or maintain a parametric gradient within the reactor body.

2. The reaction device according to claim 1, wherein the reactor body is slanted, an angle formed between the reactor body and the gravity direction of the Earth is greater than 0° but less than 90° or placed vertically, and the first end is positioned above the second end; the continuous phase is in gravity the flow is directed towards the second end under the action.

3. The reaction device according to claim 1, wherein the reactor body is placed horizontally, and the first end is provided with the second injection port, to inject a treatment liquid with an initial flow rate; the continuous phase is directed to flow toward the second end, driven by the treatment liquid.

4. The reaction device according to claim 1, wherein the first injection port is arranged in the middle, anywhere between the first end and the second end of the reactor body.

5. The reaction device according to claim 1, wherein the reactor body comprises
a first accommodating portion and a second accommodating portion;
a first joint, connecting the first accommodating portion and the second accommodating portion, one end of the first accommodating portion away from the first joint is the first end, and the first injection port is disposed in or on the first connector joint;
the second joint communicates with the second accommodating portion to form the second end, and a discharge port is provided on the second joint.

6. The reaction device according to claim 1, wherein reaction device is an embryo microsphere solidification reactor or a liposome drug-carrying reactor.

7. A microsphere preparation device, comprising:
material injection mechanism, configured to output embryo microsphere;
a reaction device according to claim 1,
wherein the first end of the reaction device is further provided with a third injection port, which communicates with the material injection mechanism, through the material injection mechanism, embryonic microspheres are injected, so that the embryonic microspheres settle in the reaction device and solidify, and form by extraction to form microspheres; and
a collector is communicated with the second end of the reaction device to collect microspheres.

8. A microsphere solidification method, for extracting the organic solvent in embryo microsphere, comprising
providing a reaction device according to claim 1, the first liquid is a microsphere receiving solution, the microsphere receiving solution uses water as a matrix to extract the organic solvent;
embryonic microspheres are moved from the first end to the second end along the reactor body containing the microsphere receiving solution, the organic solvent of the embryonic microspheres is extracted out, and the embryonic microspheres is hardened to form microspheres;
during the extraction, water is injected through the first injection port, and the water flows to the second end in the reactor body to form a concentration gradient of water on both sides of the first injection port.

9. A microsphere curing method according to claim 8, wherein
water is continuously or intermittently injected into the first injection port; and/or;
water flow is driven by gravity or a directional flow of the microsphere receiving solution; and/or;
the flow rate of water injects the first injection port is controllable.

10. A liposome drug-loading method, wherein:
providing a reaction device of claim 1,
installing a phospholipid membrane reaction device,
injecting an acidic or alkaline liquid through the first injection port to maintain a pH gradient on both sides of the phospholipase.
